(19) United States
(12) Patent Application Publication
(10) Pub. No.: US 2004/0059357 A1
(43) Pub. Date: Mar. 25, 2004

(54) Sternum suture band

(76) Inventor: Tomoaki Koseki, Chiyoda-ku (JP)

Correspondence Address: Tomoaki Koseki, 17-2, Sotokanda 2-chome, Chiyoda-ku, Tokyo, 101-0021, JP

(21) Appl. No.: 10/672,173

(22) Filed: Sep. 25, 2003

(30) Foreign Application Priority Data
Sep. 25, 2002 (JP) JP-2002-279915

(51) Int. Cl. A61B 17/08

(52) U.S. Cl. 606/151

(57) ABSTRACT

Fastening sternum requires much force, and fastening sternum with stainless wire has difficulties that when the wire is twisted too much the wire will cut off, and when the wire force is too weak, the fixation of the sternum is not sufficient. Also, using stainless steel wire means metal object are left inside the body, and this would cause halation during Magnetic Resonance Imaging photography. This invention proposes a solution for the above, by providing a sternum suture band, wherein a needle is attached on one end of a resin sash form band to pierce through the sternum, one side of the sash having a multiple number of serial projections to prevent untwining of the band, the needle is passed through the box, and then the sternum is gradually fastened and fixed. Fastening of the sternum is facilitated by using a band fastening device for the sternum suture band, wherein the band is inserted from the insertion guide, and when a slide grip is pulled, a hold gear holds one end of the band, and pulls the sternum by pulling the band to the foreside, and when the tension exceeds the pulling intensity of the band the band is automatically cut off.

### BACKGROUND OF THE INVENTION

1. Field of the Invention

This invention relates to a material for sternum suture utilized during cardiac surgery.

2. Description of the Prior Art

A common method when conducting suture on sternum is, when the sternum is pierced utilizing needle-attached wires or a gimlets, and the suture is fastened by twisting single wires.

In such cases, when the twisting of the wire is conducted too firmly, the wire breaks, and when the twisting strength is too weak, then the fixing of the sternum becomes difficult. Fastening the sternum is the biggest task at the last point of a cardiac surgery, and the fastening procedure requires much force. Also such a procedure leaves metal foreign objects inside the body. This causes halation when photographing Magnetic Resonance Imaging (MRI).

### SUMMARY OF THIS INVENTION

To solve the above issues, this invention proposes a sternum suture band, wherein, a needle to pierce through the sternum is attached to one end of a band made of resin, the center part of the band is in a sash form, and one side of the sash has a multiple number of serial projection to prevent untwining. The other end of the sash is formed into a box form, through which the needle that pierced through the sternum passes, and the lower side of the upper part of the inside of the box has receptors for the projections for prevention of untwining, and thus the sternum is fastened gradually and then fixed.

This sternum suture band enables a suture procedure conducted with a dull-end needle, not to directly pierce through the sternum but to conduct suture along the costae without damaging internal thoracic artery and other blood vessels.

This sternum suture band is made of material which is absorbed inside the body, through mechanisms of hydrolysis or decomposition by enzymes.

This sternum suture band functions to fasten the sternum when this band is fastened, wherein the sternum suture band is inserted from the insertion guide at one end, the front side of the insertion guide is closely adhered to the box part, and when the slide grip is pulled, a holding gear holds one end of the sternum suture band, fastening the sternum to the foreside, and when the tension exceeds the pulling intensity of the band, the sternum suture band is automatically cut off.

### DESCRIPTION OF THE PREFERRED EMBODIMENTS

Hereafter, a description of the preferred embodiments of this invention is made in reference to drawings. FIG. 1 shows one embodiment of this invention according to claims 1, 2 and 3, wherein a dull-end needle 2 is attached on one end of the band to pierce through the sternum, the center part of the band 1 is in a sash form, one side of the sash has a multiple number of serial projections to prevent the wire from untwining. The other end of the band is in a box form 3, and the needle that pierced through the sternum passes through this box 3, the lower part of the upper portion of the inside of this box has receptors to receive the untwining-prevention projections, and thus the sternum is fastened gradually and then fixed. The needle is made of stainless steel, and the preferred material for the band and the solid body mould box is polyethylene, polypropylene or other such bio-adaptable material. It is also effective to utilize polyglycol acid, poly lactic acid, polydioxanone and other such bio-absorbable material.

FIG. 2 shows one embodiment of this invention according to claim 4, of a fastening device while fastening the sternum suture band. The structure of the sternum suture band is made so that a sternum suture band is inserted from the insertion guide 5 at one end, the front side of the insertion guide is adhered closely with box part 3, and when the slide grip 10 is pulled, the hold gear 6 holds one end of the sternum suture band, pulls in towards the foreside to fasten the sternum, and when the tension exceeds the pulling intensity of the sternum suture band, the sternum suture band is automatically cut off.

As explained above, this invention is bio-adaptability, enabling easy and firm suture of the sternum, and as this is made of resin, no halation occurs when photographing with Magnetic Resonance Imaging.

### BRIEF DESCRIPTION OF THE DRAWINGS

FIG. 1 is an explanatory drawing of one embodiment of a sternum suture band.

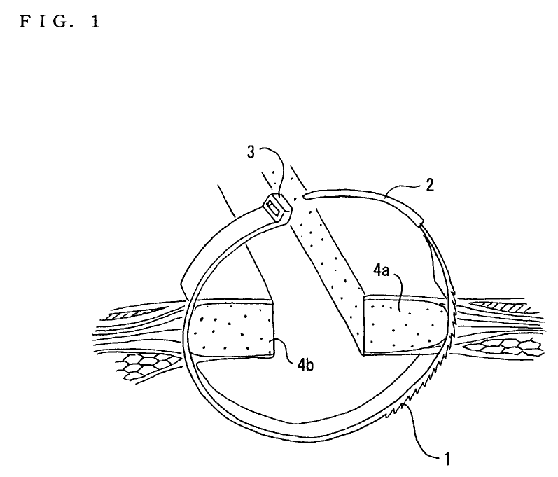

FIG. 2 is an explanatory drawing of an embodiment of a fastening device utilizing a sternum suture band.

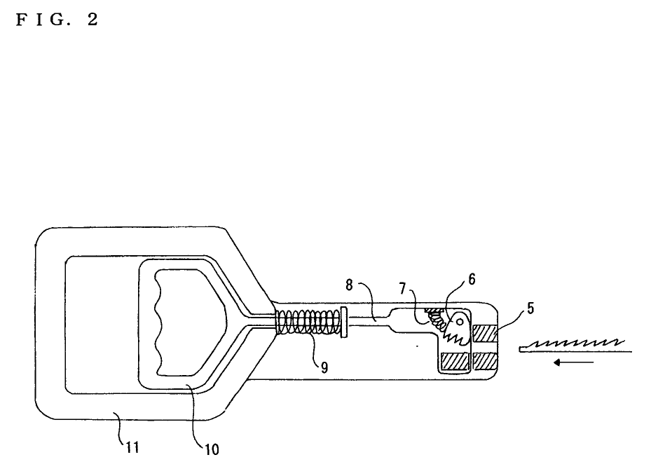

What is claimed is:

1. A sternum suture material utilized during a cardiac surgery, wherein a needle to pierce through the sternum is attached at one end of a resin-made band, the center part of the band is in a sash form, one side of the sash has a multiple number of serial projections to prevent untwining, the other end of the band is made in a box form, the needle that pierces through the sternum passes through inside the box, and the lower side of the upper portion of the inside of the box has receptors to receive the untwining-prevention projections, thus this sternum suture band is made to gradually fasten and then fix the sternum.

2. A sternum suture band according to claim 1, wherein the tip end of the needle attached to one end of the band is in a dull form, not to directly pierce through the sternum but to enable suture along the costae without damaging the internal thoracic artery and other blood vessels.

3. A sternum suture band according to claims 1 and 2, wherein the main part of the band is made of material that are absorbed in the body through mechanisms of hydrolysis or decomposition by enzymes.

4. A fastening device for fastening the sternum suture band according to claims 1, 2 and 3, wherein a sternum suture band is inserted from the insertion guide at one end of the band, the front box of the insertion is adhered closely with the box part, and when the slide grip is pulled, the hold gear holds one end of the sternum suture band, to pull in the sternum to the foreside, and when the tension exceeds the pulling intensity of the sternum suture band, the band is automatically cut off.
